(19) United States
(12) Patent Application Publication
(10) Pub. No.: US 2026/0036498 A1
(43) Pub. Date: Feb. 5, 2026

(54) PROCESS FOR DETECTION OF FETAL CELLS

(71) Applicant: Menarini Silicon Biosystems S.p.A., Bologna (IT)

(72) Inventors: Anna Doffini, Bologna (IT); Chiara Maranta, Bologna (IT); Chiara Mangano, Bologna (IT); Emilia Dora Giovannone, Bologna (IT); Thomas J. Musci, Bologna (IT)

(73) Assignee: Menarini Silicon Biosystems S.p.A., Bologna (IT)

(21) Appl. No.: 19/216,013

(22) Filed: May 22, 2025

(30) Foreign Application Priority Data
May 22, 2024 (IT) 102024000011620

(51) Int. Cl. G01N 1/30; C12Q 1/6869

(52) U.S. Cl. CPC G01N 1/30; C12Q 1/6869

(57) ABSTRACT

Provided herein is a method of isolating fetal cells from a sample from a pregnant subject. The method utilizes magnetic particles conjugated to anti-EGFR or a combination of anti-EGFR and anti-CD105 antibodies to create an enriched sample and then labeled antibodies for fetal cell markers to isolate different fetal cell types in the sample from the pregnant subject. Said isolated fetal cells can then be analyzed via gene sequencing or genomic or genetic analysis.

### CROSS REFERENCE TO RELATED APPLICATIONS

This application claims priority to and the benefit of Italian Application No. 102024000011620, filed May 22, 2024. The contents of these applications are incorporated herein by reference in their entireties.

### TECHNOLOGICAL FIELD

This disclosure relates to a method for isolating and detecting fetal cells in a sample from a pregnant subject. These isolated fetal cells can then be used in downstream gene sequencing or genomic or genetic analysis or as a diagnostic tool.

### BACKGROUND

The following description of the background of the present technology is provided simply as an aid in understanding the present technology and is not admitted to describe or constitute prior art to the present technology.

Currently, the routine clinical practices for diagnosing chromosomal abnormalities in fetuses during pregnancy include invasive procedures, such as amniocentesis and chorionic villus sampling (CVS), that pose potential risks to both the mother and fetus. Thus, the ability to isolate fetal cells and fetal DNA from maternal blood presents an exciting opportunity for improved noninvasive prenatal testing.

Recently, a cell-free DNA-based screening (cfDNA) method, known as non-invasive prenatal testing (NIPT), was introduced in prenatal screening and is recognized as highly predictive for trisomy 21. However, the screening performance of NIPT remains below that of the other more invasive diagnostic tools, necessitating the use of additional confirmatory tests. NIPT is not predictive of copy number variants (CNVs) or microdeletions and microduplications, according to professional societies (Practice bulletin n163, Obstet. Gynocol. 2016; 127 (5) 979-981).

Direct analysis of fetal cells from maternal blood samples has been challenging given the scarcity of fetal cells in maternal blood (0.1-10 fetal cells in 1 mL of maternal blood, which contains about 1-5 million cells). Many methods of enrichment, such as filters, density gradients, fluorescence activated cell sorting, microfluidics, and immuno-magnetic bears, have been tested, but all have lacked consistency and reproducibility although circulating fetal cells have been recovered. The major challenge is to eliminate all the contaminating nucleated blood cells without losing the few circulating fetal cells in the first trimester of gestation. Taking these limitations and the risks posed by the more effective but invasive diagnostic tools into account, there is a need to develop a new cell-based non-invasive prenatal diagnosis procedure to select fetal cells from the blood of pregnant women to screen for birth defects and inherited diseases.

Disclosed herein are fetal cell markers and agents that bind them. Further disclosed herein are compositions and methods for isolating fetal cells based on said fetal cell markers.

### SUMMARY

The present disclosure provides novel approaches to isolating fetal cells by exposing samples from pregnant subjects to a combination of ligands and/or receptor proteins associated with human extravillous trophoblasts (EVTs). As shown herein, the molecules used are epidermal growth factor receptor (EGFR) alone, which is sufficient to isolate EVTs from samples, or in combination with CD105, which can improve isolation of EVTs. It is to be understood that the disclosed embodiments are merely exemplary, and accordingly, the invention may be embodied in various and alternative forms. The specific structural and functional details disclosed herein are not to be interpreted as limiting but merely as a basis for the claims and as a representative basis for teaching one skilled in the art to variously employ the embodiments described herein.

In a first aspect, the present disclosure provides a method for isolating fetal cells from a sample from a pregnant subject, comprising (a) contacting the sample comprising a plurality of cells with a plurality of magnetic particles, the magnetic particles conjugated to an anti-epidermal growth factor receptor (EGFR) antibody; (b) isolating cells bound to the anti-EGFR antibody by subjecting the sample to a magnetic field, thereby producing an enriched sample; (c) contacting the enriched sample with a first labeled antibody that binds to a cytokeratin (CK); and (d) isolating a fetal cell that is bound to the first labeled antibody.

In some embodiments according to this first aspect, the fetal cell is a fetal nucleated red blood cell (fnRBC). In some embodiments of this first aspect, the fetal cell is a trophoblast. In some further embodiments of this first aspect, the trophoblast is an extravillous cytotrophoblast (cEVT). In some embodiments of this first aspect, the magnetic particles are colloidal magnetic particles. In some further embodiments according to this first aspect, the colloidal magnetic particles are ferrofluid magnetic particles.

In some embodiments according to this first aspect, the magnetic particles are coupled to a first exogenous aggregation enhancing factor (EAEF), the first EAEF being one member of a specific binding pair selected from the group including biotin-streptavidin, antigen-antibody, receptor-hormone, receptor-ligand, agonist-antagonist, lectin-carbohydrate, Protein A-antibody Fc, avidin-biotin, biotin analog-avidin, desthiobiotin-streptavidin, desthiobiotin-avidin, iminobiotin-streptavidin, and iminobiotin-avidin. In some further embodiments of this first aspect, the method includes adding a second EAEF to induce aggregation of the magnetic particles, wherein the second EAEF is the other member of the specific binding pair. In some further embodiments of this first aspect, the method includes adding to the enriched sample a member of the specific binding pair in order to reverse aggregation of the magnetic particle in the enriched sample.

In some embodiments according to this first aspect, the method further involves contacting the enriched sample with a second labeled antibody that binds CD45. In some embodiments according to this first aspect, the first labeled antibody is a fluorescent label. In some embodiments of this first aspect, the second labeled antibody is a fluorescent label. In some embodiments of this first aspect, isolating the fetal cells includes using immunofluorescent technology, fluorescence activated cell sorting (FACS), or DEPArray. In some embodiments of this first aspect, the method further involves gene sequencing of the fetal cell. In some embodiments according to this first aspect, the method further involves performing a genomic or a genetic analysis of the fetal cell.

In a second aspect, the present disclosure provides a method for isolating fetal cells from a sample from a pregnant subject, including a) contacting the sample comprising a plurality of cells with a plurality of magnetic particles, wherein each magnetic particle in the plurality is conjugate to an anti-EGFR antibody; b) subjecting the sample to a magnetic field; c) isolating cells bound to the magnetic particles to produce an enriched sample; d) contacting the enriched sample with: i) an antibody that binds to cytokeratin (CK) and is conjugated to a fluorescent label, ii) an antibody that binds to CD45 and is conjugated to a fluorescent label, and iii) a nuclear stain; and d) isolating a fetal cell using fluorescence activated cell sorting (FACS) or DEPArray, wherein the fetal cell is positive for CK and nuclear stain and negative for CD45.

In some embodiments of this second aspect, the fetal cell is a fetal nucleated red blood cell (fnRBC). In some embodiments according to this second aspect, the fetal cell is a trophoblast. In some further embodiments of this second aspect, the trophoblast is an extravillous cytotrophoblast (cEVT). In some embodiments of this second aspect, the method further includes genetic sequencing of the fetal cell. In some embodiments of this second aspect, the method further includes performing a genomic or genetic analysis of the fetal cell.

In some embodiments according to this second aspect, the magnetic particles are colloidal magnetic particles. In some further embodiments of this second aspect, the colloidal magnetic particles are ferrofluid magnetic particle.

In a third aspect, the present disclosure provides a method for isolating fetal cells from a sample from a pregnant subject, involving: a) contacting a sample including an anti-epidermal growth factor receptor (EGFR) antibody; b) isolating cells bound to the anti-EGFR antibody, thereby producing an enriched sample; c) contacting the enriched sample with a first labeled antibody that binds to a fetal cell marker; and d) isolating a fetal cell that is bound to the first labeled antibody.

In some embodiments according to this third aspect, the fetal cell marker is a cytokeratin (CK). In some embodiments of this third aspect, the fetal cell is a fetal nucleated red blood cell (fnRBC) or a trophoblast. In some embodiments of this third aspect, the anti-EGFR antibody is conjugated to a magnetic particle. In further embodiments of this third aspect, the magnetic particle is a colloidal magnetic particle, which is optionally a ferrofluid magnetic particle. In some aspects of this third aspect, isolating cells bound to the anti-EGFR antibody involves subjecting the sample to a magnetic field. In some embodiments of this aspect, contacting the enriched sample with a first labeled antibody that binds to a fetal cell marker includes contacting the enriched sample with a second labeled antibody that binds CD45. In some embodiments of this third aspect, isolating the fetal cell includes using immunofluorescent technology, fluorescence activated cell sorting (FACS), or DEPArray. In some embodiment according to this third aspect, the method further involves gene sequencing of the fetal cell or involves performing a genomic or a genetic analysis of the fetal cell.

In a fourth aspect, the present disclosure provides a method for isolating fetal cells from a sample from a pregnant subject, comprising: (a) contacting the sample comprising a plurality of cells with a plurality of magnetic particles, wherein the plurality of magnetic particles comprises: (i) magnetic particles that are conjugated to an anti-epidermal growth factor receptor (EGFR) antibody and an anti-CD105 antibody, or (ii) a combination of magnetic particles that are conjugated to an anti-EGFR antibody and magnetic particles that are conjugated to an anti-CD105 antibody; (b) isolating cells bound to the first antibody or the second antibody by subjecting the sample to a magnetic field, thereby producing an enriched sample; (c) contacting the enriched sample with a first labeled antibody that binds to a cytokeratin (CK); and (d) isolating a fetal cell that is bound to the first labeled antibody.

In some embodiments according to this fourth aspect, the fetal cell is a fetal nucleated red blood cell (fnRBC). In some embodiments of this first aspect, the fetal cell is a trophoblast. In some further embodiments of this fourth aspect, the trophoblast is an extravillous cytotrophoblast (cEVT). In some embodiments of this fourth aspect, the magnetic particles are colloidal magnetic particles. In some further embodiments according to this fourth aspect, the colloidal magnetic particles are ferrofluid magnetic particles.

In some embodiments according to this fourth aspect, the magnetic particles are coupled to a first exogenous aggregation enhancing factor (EAEF), the first EAEF being one member of a specific binding pair selected from the group including biotin-streptavidin, antigen-antibody, receptor-hormone, receptor-ligand, agonist-antagonist, lectin-carbohydrate, Protein A-antibody Fc, avidin-biotin, biotin analog-avidin, desthiobiotin-streptavidin, desthiobiotin-avidin, iminobiotin-streptavidin, and iminobiotin-avidin. In some further embodiments of this fourth aspect, the method includes adding a second EAEF to induce aggregation of the magnetic particles, wherein the second EAEF is the other member of the specific binding pair. In some further embodiments of this fourth aspect, the method includes adding to the enriched sample a member of the specific binding pair in order to reverse aggregation of the magnetic particle in the enriched sample.

In some embodiments according to this fourth aspect, the method further involves contacting the enriched sample with a second labeled antibody that binds CD45. In some embodiments according to this fourth aspect, the first labeled antibody is a fluorescent label. In some embodiments of this fourth aspect, the second labeled antibody is a fluorescent label. In some embodiments of this fourth aspect, isolating the fetal cells includes using immunofluorescent technology, fluorescence activated cell sorting (FACS), or DEPArray. In some embodiments of this fourth aspect, the method further involves gene sequencing of the fetal cell. In some embodiments according to this fourth aspect, the method further involves performing a genomic or a genetic analysis of the fetal cell.

In a fifth aspect, the present disclosure provides a method for isolating fetal cells from a sample from a pregnant subject, comprising: (a) contacting the sample comprising a plurality of cells with a plurality of magnetic particles, wherein the plurality of magnetic particles comprises: (i) magnetic particles that are conjugated to an anti-epidermal growth factor receptor (EGFR) antibody and an anti-CD105 antibody, or (ii) a combination of magnetic particles that are conjugated to an anti-EGFR antibody and magnetic particles that are conjugated to an anti-CD105 antibody; (b) subjecting the sample to a magnetic field; (c) isolating cells bound to the magnetic particles to produce an enriched sample; (d) contacting the enriched sample with: (i) an antibody that binds to a cytokeratin (CK) and is conjugated to a fluorescent label, (ii) an antibody that binds to CD45 and is conjugated to a fluorescent label, and (iii) a nuclear stain; and (d) isolating a fetal cell using fluorescence activated cell sorting (FACS) or DEPArray, wherein the fetal cell is positive for the CK and nuclear stain and negative for CD45.

In some embodiments of this fifth aspect, the fetal cell is a fetal nucleated red blood cell (fnRBC). In some embodiments according to this fifth aspect, the fetal cell is a trophoblast. In some further embodiments of this fifth aspect, the trophoblast is an extravillous cytotrophoblast (CEVT). In some embodiments of this fifth aspect, the method further includes genetic sequencing of the fetal cell. In some embodiments of this fifth aspect, the method further includes performing a genomic or genetic analysis of the fetal cell.

In some embodiments according to this fifth aspect, the magnetic particles are colloidal magnetic particles. In some further embodiments of this fifth aspect, the colloidal magnetic particles are ferrofluid magnetic particle.

In a sixth aspect, the present disclosure provides a method for isolating fetal cells from a sample from a pregnant subject, comprising: (a) contacting the sample comprising an anti-epidermal growth factor receptor (EGFR) antibody and an anti-CD105 antibody; (b) isolating cells bound to the anti-EGFR antibody and/or anti-CD105 antibody, thereby producing an enriched sample; (c) contacting the enriched sample with a first labeled antibody that binds to a fetal cell marker; and (d) isolating a fetal cell that is bound to the first labeled antibody.

In some embodiments according to this sixth aspect, the fetal cell marker is a cytokeratin (CK). In some embodiments of this sixth aspect, the fetal cell is a fetal nucleated red blood cell (fnRBC) or a trophoblast. In some embodiments of this sixth aspect, the anti-EGFR antibody is conjugated to a magnetic particle. In further embodiments of this sixth aspect, the magnetic particle is a colloidal magnetic particle, which is optionally a ferrofluid magnetic particle. In some aspects of this sixth aspect, isolating cells bound to the anti-EGFR antibody involves subjecting the sample to a magnetic field. In some embodiments of this aspect, contacting the enriched sample with a first labeled antibody that binds to a fetal cell marker includes contacting the enriched sample with a second labeled antibody that binds CD45. In some embodiments of this sixth aspect, isolating the fetal cell includes using immunofluorescent technology, fluorescence activated cell sorting (FACS), or DEPArray. In some embodiment according to this sixth aspect, the method further involves gene sequencing of the fetal cell or involves performing a genomic or a genetic analysis of the fetal cell.

### BRIEF DESCRIPTION OF THE DRAWINGS

FIGS. 1A, 1B, 1C, 1D, and 1E depict an exemplary workflow for the described method.

FIG. 2 shows the number of cEVTs recovered in samples from all subjects using either magnetic particles conjugated with anti-CD105 and anti-HER2 antibodies or magnetic particles conjugated with the anti-EGFR antibody.

FIG. 3 shows the number of cEVTs recovered in samples from positive subjects using either magnetic particles conjugated with anti-CD105 and anti-HER2 antibodies or magnetic particles conjugated with the anti-EGFR antibody.

FIG. 4 shows the cEVT recovery percentage using either magnetic particles conjugated with anti-CD105 and anti-HER2 antibodies or magnetic particles conjugated with the anti-EGFR antibody.

FIG. 5 shows the number of cEVTs recovered in samples from all subjects using either magnetic particles conjugated with anti-CD105 antibody or magnetic particles conjugated with anti-EGFR antibody.

FIG. 6 shows the cEVT recovery percentage based on STR analysis using either magnetic particles conjugated with anti-CD105 antibody or magnetic particles conjugated with the anti-EGFR antibody.

FIG. 7 shows the number of cEVTs recovered in samples from all subjects using either magnetic particles conjugated with anti-CD105 antibody or a mixture of magnetic particles conjugated with anti-EGFR antibody and magnetic particles conjugated with anti-CD105 antibody.

FIG. 8 shows the cEVT recovery percentage using either magnetic particles conjugated with anti-CD105 antibody or a mixture of magnetic particles conjugated with anti-EGFR antibody and magnetic particles conjugated with anti-CD105 antibody.

FIG. 9 shows the number of cEVTs recovered in samples from all subjects using either a mixture of magnetic particles conjugated with anti-CD105 antibody and magnetic particles conjugated with anti-EGFR or a mixture of magnetic particles conjugated with anti-CD105 antibody and magnetic particles conjugated with anti-HER2 antibody.

FIG. 10 shows the cEVT recovery percentage using either a mixture of magnetic particles conjugated with anti-CD105 antibody and magnetic particles conjugated with anti-EGFR or a mixture of magnetic particles conjugated with anti-CD105 antibody and magnetic particles conjugated with anti-HER2 antibody.

FIG. 11 shows the number of cEVTs recovered in 20 mL maternal blood samples from all subjects using either magnetic particles conjugated with anti-CD105 antibody and anti-EGFR or a mixture of magnetic particles conjugated with anti-CD105 antibody and magnetic particles conjugated with anti-EGFR.

FIG. 12 shows the cEVT recovery percentage based on STR analysis using either magnetic particles conjugated with anti-CD105 antibody and anti-EGFR or a mixture of magnetic particles conjugated with anti-CD105 antibody and magnetic particles conjugated with anti-EGFR in 20 mL maternal blood samples.

FIG. 13 shows the number of cEVTs recovered in 30 mL maternal blood samples from all subjects using either magnetic particles conjugated with anti-CD105 antibody and anti-EGFR or a mixture of magnetic particles conjugated with anti-CD105 antibody and magnetic particles conjugated with anti-EGFR.

FIG. 14 shows the cEVT recovery percentage using either magnetic particles conjugated with anti-CD105 antibody and anti-EGFR or a mixture of magnetic particles conjugated with anti-CD105 antibody and magnetic particles conjugated with anti-EGFR in 30 mL maternal blood.

FIG. 15 shows examples of particles that can be used in the disclosed methods.

### DETAILED DESCRIPTION

The functional unit of the placenta is the villus, which is composed of epithelial trophoblasts differentiated from the trophectoderm and a stromal cell core that contains fetal endothelial cells, Hofbauer cells, and mesenchymal stroma cells, among others. The inner layer of the trophoblast is made up of cytotrophoblasts (CTBs), which express receptors involved in cellular proliferation and differentiation. These receptors include epidermal growth factor receptor (EGFR), neuropilin-2 (NRP2), and hepatocyte growth factor receptor (HGFR). These receptors are thought to interact with Hofbauer cells and placental fibroblast cells. In the CTB, there are two distinct differentiation pathways to generate the other type types of epithelial trophoblasts, which are syncytiotrophoblasts (STBs) and extravillous trophoblasts (EVTs). STBs are responsible for producing placental hormones and scarcely express any major histocompatibility complex (MHC) class I or class II molecules, which are also known as human leukocyte antigen (HLA). This allows these cells to avoid immune attack mediated by allogenic recognition from T cells. EVTs are in charge of invading the decidua and participating in spiral arteries remodeling. These cells express HLA-C, HLA-E, and HLA-G, along with receptors involved in immune regulation including atypical chemokine receptor 2 (ACKR2) and C-X-C chemokine receptor type 6 (CXCR6). The important maternal-fetal interface is composed of EVTs, decidual stromal cells (DSCs), and decidual immune cells. Fetal nucleated red blood cells (fnRBCs) and trophoblastic cells originally from the placenta can be present in the maternal circulation.

The present disclosure provides methods that, first, increase the number of isolated cEVTs per subject as different trophoblast subtypes could be present in a given subject and, second, to target differentially differentiated cEVTs. These methods utilize markers for different subtypes to increase the likelihood of detecting trophoblasts that fall within said subtypes.

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein for the purpose of describing particular embodiments only and is not intended to be limiting.

### I. Definitions

Unless defined otherwise, all technical and scientific terms used herein generally have the same meaning as commonly understood by one of ordinary skill in the art to which this technology belongs. As used in this specification and the appended claims, the singular forms “a,” “an,” and “the” include plural referents unless the context clearly dictates otherwise. For example, reference to a “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “peptide” refers to a polymer of amino acid residues joined by amide linkages, which may optionally be chemically modified to achieve desired characteristics. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include unnatural amino acids or residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

As used herein, the terms “subject,” “patient,” or “individual” may be used interchangeably throughout and can be an individual organism, a vertebrate, a mammal, or a human. In some embodiments, the subject, patient or individual is a human, specifically a pregnant human.

A “fragment” is a portion of an amino acid sequence or a polynucleotide which is identical in sequence to but shorter in length than a reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 4, 5, 6, 7, 8, 9, 10, 11, 12, 13, 14, 15, or 16 contiguous amino acid residues of a reference peptide, respectively. Fragments may be preferentially selected from certain regions of a molecule. The term encompasses the full-length polynucleotide or full-length polypeptide.

### II. Epidermal Growth Factor Receptor (EGFR)

Epidermal growth factor receptor (EGFR) is a transmembrane glycoprotein that is a member of the protein kinase superfamily. It is a receptor for members of the epidermal growth factor family. EGFR is a cell surface protein that binds to epidermal growth factor, inducing receptor dimerization and tyrosine autophosphorylation leading to cell proliferation. Several studies reported EGFR expression in trophoblasts, especially in proliferating ones. Reduced expression of EGFR is observed in many pregnancy-related complications including preeclampsia (PE), intrauterine growth restriction (IUGR), and recurrent spontaneous abortion. EGFR overexpression is seen in hydatidiform mole and choriocarcinoma. Recently, single-cell RNA sequencing studies showed the expression of EGFR gene in different trophoblast subsets, including villous cytotrophoblasts, syncytiotrophoblasts, and extravillous trophoblasts.

The first antibody may be an antibody that binds to EGFR protein. The antigen binding fragment may bind to EGFR protein. The first antibody or antigen binding fragment thereof may comprise, consist of, or consist essentially of any one of the anti-EGFR antibody sequences disclosed herein. However, any anti-EGFR antibody could be used for the purposes of the present disclosure. Those skilled in the art will understand that the particular anti-EGFR antibody that is used is not critical, so long as the anti-EGFR antibody specifically binds to EGFR. Anti-EGFR antibodies that could be used for the purposes of the present disclosure include, but are not limited to, commercially available anti-EGFR antibodies from Abcam (e.g., Cat. No. ab52894, Cat. No. ab32077, Cat. No. ab289889), Invitrogen (e.g., Cat. No. MA5-13319, Cat. No. PA1-1110, Cat. No. MA5-13269), and GeneTex (e.g., Cat. No. GTX628887, Cat. No. GTX121919).

The first antibody or antigen binding fragment thereof that binds to EGFR may be coupled to a magnetic particle. For instance, the first antibody or antigen binding fragment thereof may be in the form of a ferrofluid. Isolating the cells that are bound to the first antibody or antigen binding fragment thereof that binds to EGFR may comprise any of the cell isolation techniques disclosed herein. Isolating the cells may comprise magnetic separation. Isolating the cells may comprise EAEF-based aggregation and reversal.

### III. HER2

HER2 (also known as Neu and ERBB2). It does not bind to a ligand but can dimerize and recruit the largest group of phosphotyrosine-binding proteins among all ERBB members. Previous studies reported the induction of HER2 in human extravillous trophoblasts (EVTs), especially in invasive EVTs (iEVTs). Development of the EVT lineage shares many features with early phases of tumor formation. During EVT differentiation, many markers are expressed, including HLA-G, T-cell factor 4, integrin α5 and β1, Notch2, proteoglycan 2, and ErbB2. Additionally, ERBB2-ERBB3 axis stimulation by exogenous neuregulin seems to be involved in trophoblast survival during human placental development. Recently, single-cell RNA sequencing studies showed the expression of HER2 in different trophoblast subsets, including iEVTs.

The second antibody may comprise an antibody that binds to HER2 protein. The antigen binding fragment may bind to HER2 protein. The first antibody or antigen binding fragment thereof may comprise, consist of, or consist essentially of any one of the anti-HER2 antibody sequences disclosed herein. However, any anti-HER2 antibody could be used for the purposes of the present disclosure. Those skilled in the art will understand that the particular anti-HER2 antibody that is used is not critical, so long as the anti-HER2 antibody specifically binds to HER2. Anti-HER2 antibodies that could be used for the purposes of the present disclosure include, but are not limited to, commercially available anti-HER2 antibodies from Abcam (e.g., Cat. No. ab134182, Cat. No. ab237715, Cat. No. ab214275), Invitrogen (e.g., Cat. No. AHO1011, Cat. No. MA5-13679, Cat. No. MA1-12691), and GeneTex (e.g., Cat. No. GTX100509, Cat. No. GTX22428).

The first antibody or antigen binding fragment thereof that binds HER2 protein may be coupled to a magnetic particle. For instance, the first antibody or antigen binding fragment thereof may that binds to HER2 protein be in the form of a ferrofluid. Isolating the cells that are bound to the second antibody or antigen binding fragment thereof may comprise any of the cell isolation techniques disclosed herein. Isolating the cells can include magnetic separation. Isolating the cells can include EAEF-based aggregation and reversal.

### IV. CD105

CD105 (also known as endoglin, ENG, END, FLJ41744, HHT1, ORW, and ORW1) is a type I membrane glycoprotein located on cell surfaces and is part of the TGF beta receptor complex. While CD105 expression is initially low in resting endothelial cells, it has an important in the development of new blood vessels (neoangiogenesis) in places like tumor vessels, inflamed tissues, skin with psoriasis, vascular injury and during embryogenesis. During embryogenesis, CD105 is found on the vascular endothelium of human embryos during all developmental stages after 4 weeks, and it is transiently upregulated on cushion tissue mesenchyme during heart septation. Recently, immunohistochemical staining of placental tissues confirmed CD105 staining in EVTs and 76% of fetal cells enriched by CD105 were found to be cytokeratin-positive.

The second antibody may comprise an antibody that binds to CD105 protein. The antigen binding fragment may bind to CD105 protein. The second antibody or antigen binding fragment thereof may comprise, consist of, or consist essentially of any one of the anti-CD105 antibody sequences disclosed herein. However, any anti-CD105 antibody could be used for the purposes of the present disclosure. Those skilled in the art will understand that the particular anti-CD105 antibody that is used is not critical, so long as the anti-CD105 antibody specifically binds to CD105. Anti-CD105 antibodies that could be used for the purposes of the present disclosure include, but are not limited to, commercially available anti-CD105 antibodies from Abcam (e.g., Cat. No. ab221675, Cat. No. ab268052, Cat. No. ab156756, Cat. No. ab69772, Cat. No. ab114052, Cat. No. ab11414, and Cat. No. ab21224), Thermo Fisher Scientific (e.g., Cat. No. MA5-17041, Cat. No. MA1-19231, Cat. No. 16-1057-82, Cat. No. 14-1057-82, Cat. No. 14-1051-82, Cat. No. MA5-35197, Cat. No. MA5-29697, Cat. No. MA5-53462, Cat. No. MA5-23894, Cat. No. MA5-17943, Cat. No. MHCD10500, Cat. No. PA5-12511, Cat. No. MA1-19408, Cat. No. 67075-1-IG, Cat. No. 10862-1-AP, Cat. No. 28117-1-AP, Cat. No. MA5-44848, Cat. No. MA5-29234, Cat. No. MA5-48011, Cat. No. PA5-79203, Cat. No. PA5-46971, Cat. No. PA5-111623, Cat. No. PA5-29555, Cat. No. PA5-27205, Cat. No. PA5-117933, Cat. No. PA5-29554, Cat. No. PA5-95428, Cat. No. PA5-85895, Cat. No. 98013-2-RR, Cat. No. 82993-5-RR, Cat. No. 98013-1-RR, Cat. No. 2022-RBM11-P1, Cat. No. 2022-RBM11-P1ABX, Cat. No. 2022-RBM13-P1, Cat. No. 2022-RBM13-P1ABX, Cat. No. 82993-6-RR, Cat. No. 2022-MSM6-P0, Cat. No. 2022-MSM6-P1ABX, Cat. No. 2022-MSM5-P0, Cat. No. 2022-MSM5-P1ABX, Cat. No. HA600077, Cat. No. 65048-1-IG500UG, Cat. No. H00002022-M01, Cat. No. H00002022-M26, Cat. No. 2022-MSM1-P0, Cat. No. 2022-MSM1-P1ABX, Cat. No. 2022-MSM2-P0, Cat. No. 2022-MSM2-P1ABX, Cat. No. 2022-MSM10-P1, Cat. No. 2022-MSM10-P1ABX, Cat. No. 604-650, 30730-1-AP, Cat. No. BS-0579R, Cat. No. BS-4609R, Cat. No. HA500266, Cat. No. 500-4234, Cat. No. BS-34063R, Cat. No. TA590810, Cat. No. MA5-43087, Cat. No. MA5-43088, Cat. No. MA5-48012, Cat. No. H00002022-M16, Cat. No. H00002022-M22, Cat. No. H00002022-M22, Cat. No. H00002022-M30, and Cat. No. BS-10662R), and GeneTex (e.g., Cat. No. GTX11414, Cat. No. GTX00572, Cat. No. GTX100508, Cat. No. GTX112685, Cat. No. GTX78408, Cat. No. GTX112684, Cat. No. GTX60452, Cat. No. GTX22529, Cat. No. GTX34454, Cat. No. GTX53121, and Cat. No. GTX19672).

The second antibody or antigen binding fragment thereof that binds CD105 protein may be coupled to a magnetic particle. For instance, the second antibody or antigen binding fragment thereof that binds CD105 protein may be in the form of a ferrofluid. Isolating the cells that are bound to the second antibody or antigen binding fragment thereof that binds CD105 protein may comprise any of the cell isolation techniques disclosed herein. Isolating the cells can include magnetic separation. Isolating the cells can include EAEF-based aggregation and reversal.

### V. Method for Isolating Fetal Cells

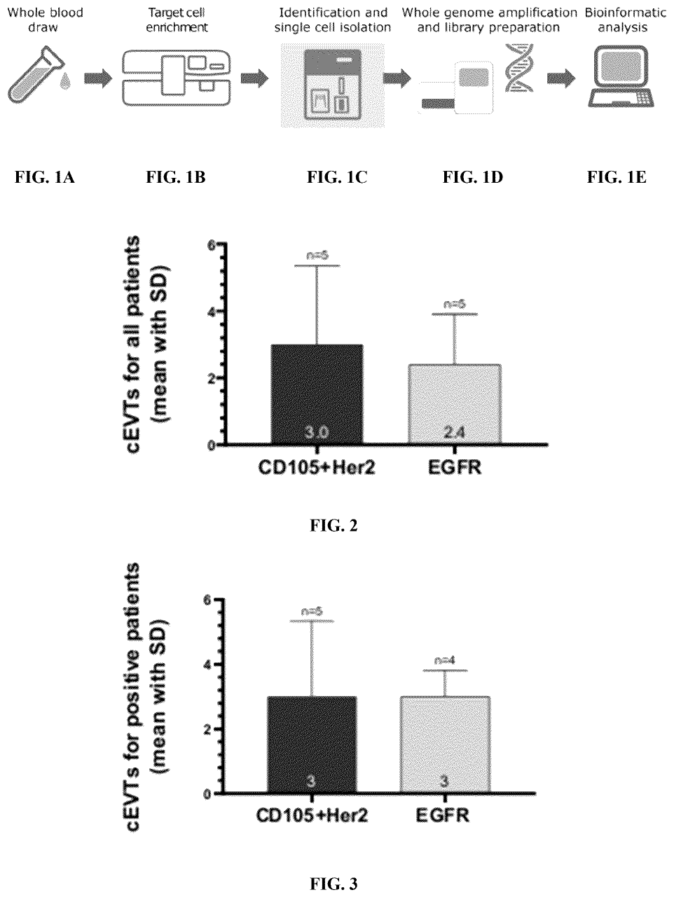

Disclosed herein are the methods for isolating fetal cells. The present disclosure provides a method of isolating fetal cells from a pregnant subject, comprising contacting the sample with a plurality of magnetic particles conjugated to antibodies for markers of interest, isolating the bound cells to produce an enriched sample, contacting the enriched sample with a labeled antibody that binds a fetal cell marker, and isolating the cells bound by the fluorescently labeled antibodies (FIGS. 1A-C). The isolated cells can then be utilized for various analyses (FIGS. 1D-E). The disclosed methods are in vitro or ex vivo methods that can be performed on a sample (e.g., a blood sample) taken from or obtained from a subject or patient (i.e., a pregnant woman).

The fetal cell may comprise a fetal nucleated red blood cell (fnRBC). The fetal cell may comprise a trophoblast. Generally, the methods comprise using an anti-EGFR antibody or antigen binding fragment thereof and, optionally, an anti-CD105 antibody or antigen binding fragment thereof to identify a cell as a fetal cell. Alternatively, or additionally the methods may comprise using said antibodies conjugated to a colloidal magnetic particle for isolating a fetal cell.

Disclosed herein is a method for isolating fetal cells in a sample from a pregnant subject, comprising a) contacting the sample comprising a plurality of cells with a plurality of magnetic particles, wherein the plurality of magnetic particles is conjugated to an anti-epidermal growth factor receptor (EGFR) antibody and b) isolating cells that are bound to the anti-EGFR. Further disclosed herein is a method for isolating fetal cells in a sample from a pregnant subject, comprising a) contacting the sample comprising a plurality of cells with a plurality of magnetic particles, wherein the plurality of magnetic particles comprises: i) magnetic particles that are conjugated to an anti-epidermal growth factor receptor (EGFR) antibody and an anti-CD105 antibody, or ii) a combination of magnetic particles that are conjugated to an anti-EGFR antibody and magnetic particles that are conjugated to an anti-CD105 antibody; and b) isolating cells that are bound to the anti-EGFR and/or anti-CD105 antibody.

Additionally disclosed herein is a method for isolating fetal cells in a sample from a pregnant subject, comprising: a) contacting the sample comprising a plurality of cells with a plurality of magnetic particles, wherein the plurality of magnetic particles comprises magnetic particles that are conjugated to an anti-epidermal growth factor receptor (EGFR); b) isolating cells bound to the anti-EGFR antibody by subjecting the sample to a magnetic field, thereby producing an enriched sample; c) contacting the enriched sample with a first labeled antibody that binds to cytokeratin (CK); and d) isolating a fetal cell that is bound to the first labeled antibody. In some embodiments, the method for isolating fetal cells in a sample from a pregnant subject, comprises: a) contacting the sample comprising a plurality of cells with a plurality of magnetic particles, wherein the plurality of magnetic particles comprises: i) magnetic particles that are conjugated to an anti-epidermal growth factor receptor (EGFR) antibody and an anti-CD105 antibody, or ii) a combination of magnetic particles that are conjugated to an anti-EGFR antibody and magnetic particles that are conjugated to an anti-CD105 antibody; b) isolating cells bound to the anti-EGFR antibody or the anti-CD105 antibody by subjecting the sample to a magnetic field, thereby producing an enriched sample; c) contacting the enriched sample with a first labeled antibody that binds to cytokeratin (CK); and d) isolating a fetal cell that is bound to the first labeled antibody.

Further disclosed herein is a method for isolating fetal cells in a sample from a pregnant subject, comprising: a) contacting the sample with a magnetic reagent and a second exogenous aggregation enhancing factor (EAEF), wherein the sample comprises a plurality of cells, wherein the magnetic reagent comprises a colloidal magnetic particle conjugated to a first antibody or antigen binding fragment thereof and/or a second antibody or antigen binding fragment thereof, wherein the colloidal magnetic particle is conjugated to a first EAEF, and wherein the first antibody or antigen binding fragment binds to EGFR, and wherein the second antibody or antigen binding fragment binds to CD105; b) contacting the sample with a first labeled antibody or antigen binding fragment thereof; and c) isolating the fetal cell that is bound to the first labeled antibody. The first EAEF may comprise a first member of a specific binding pair and the second EAEF comprises a second member of the specific binding pair, wherein the specific binding pair is selected from the group comprising biotin-streptavidin. antigen-antibody, receptor-hormone, receptor-ligand, agonist-antagonist, lectin-carbohydrate, Protein A-antibody Fc. and avidin-biotin, biotin analog-avidin, desthiobiotin-streptavidin, desthiobiotin-avidin, iminobiotin-streptavidin, and iminobiotin-avidin.

Further disclosed herein in a method for isolating fetal cells in a sample from a pregnant subject, comprising: a) contacting the sample comprising a plurality of cells with a plurality of magnetic particles, wherein the plurality of magnetic particles are conjugated to an anti-EGFR; b) subjecting the sample to a magnetic field; c) isolating cells bound to the magnetic particles to produce an enriched sample; d) contacting the enriched sample with: i) an antibody that binds to a CK and is conjugated to a first fluorescent label, ii) an antibody that binds to CD45 and is conjugated to a second fluorescent label, and iii) a nuclear stain; and d) isolating a fetal cell using fluorescence activated cell sorting (FACS) or DEPArray, wherein the fetal cell is positive for the CK and nuclear stain and negative for CD45. In some embodiments, the method for isolating fetal cells in a sample from a pregnant subject, comprises: a) contacting the sample comprising a plurality of cells with a plurality of magnetic particles, wherein the plurality of magnetic particles comprises: i) magnetic particles that are conjugated to an anti-EGFR antibody and an anti-CD105 antibody, or ii) a combination of magnetic particles that are conjugated to an anti-EGFR antibody and magnetic particles that are conjugated to an anti-CD105 antibody; b) subjecting the sample to a magnetic field; c) isolating cells bound to the magnetic particles to produce an enriched sample; d) contacting the enriched sample with: i) an antibody that binds to a CK and is conjugated to a first fluorescent label, ii) an antibody that binds to CD45 and is conjugated to a second fluorescent label, and iii) a nuclear stain; and d) isolating a fetal cell using fluorescence activated cell sorting (FACS) or DEPArray, wherein the fetal cell is positive for the CK and nuclear stain and negative for CD45.

Further disclosed herein is a method for isolating fetal cells in a sample from a pregnant subject, comprising: a) contacting the sample comprising an anti-epidermal growth factor receptor (EGFR) antibody, b) isolating cells bound to the anti-EGFR antibody, thereby producing an enriched sample; c) contacting the enriched sample with a first labeled antibody that binds to a fetal cell marker; and d) isolating a fetal cell that is bound to the first labeled antibody. Further disclosed herein is a method for isolating fetal cells in a sample from a pregnant subject, comprising: a) contacting the sample comprising an anti-epidermal growth factor receptor (EGFR) antibody and an anti-CD105 antibody, b) isolating cells bound to the anti-EGFR antibody or the anti-CD105 antibody, thereby producing an enriched sample; c) contacting the enriched sample with a first labeled antibody that binds to a fetal cell marker; and d) isolating a fetal cell that is bound to the first labeled antibody.

Further disclosed herein is a method for isolating fetal cells in a sample from a pregnant subject, comprising: a) contacting the sample with a first antibody conjugate, wherein the sample comprises a plurality of cells, and wherein the first antibody conjugate comprises a first antibody or antigen binding fragment thereof conjugated to a colloidal magnetic particle, b) isolating cells bound to the first antibody conjugate by subjecting the sample to a magnetic field, thereby producing an enriched sample, c) contacting the enriched sample with a second antibody or antigen binding fragment thereof, wherein the second antibody binds to a fetal cell marker and is labeled, and d) isolating a fetal cell that is bound to the labeled second antibody. The first antibody may comprise an antibody or antigen binding fragment thereof that binds EGFR. The first labeled antibody may bind CK.

Further disclosed herein is a method for isolating fetal cells in a sample from a pregnant subject, comprising: a) contacting the sample with a first antibody conjugate and a second antibody conjugate, wherein the sample comprises a plurality of cells, and wherein the first antibody conjugate comprises a first antibody or antigen binding fragment thereof and/or the second antibody conjugate comprises a second antibody or antigen binding fragment conjugated to a colloidal magnetic particle, b) isolating cells bound to the first antibody conjugate and/or second antibody conjugate by subjecting the sample to a magnetic field, thereby producing an enriched sample, c) contacting the enriched sample with a first labeled antibody or antigen binding fragment thereof, wherein the first labeled antibody binds to a fetal cell marker and is labeled, and d) isolating a fetal cell that is bound to the first labeled antibody. The first antibody conjugate may comprise an antibody or antigen binding fragment thereof that binds EGFR. The second antibody conjugate may comprise an antibody or antigen binding fragment thereof that binds CD105. The first labeled antibody may comprise and antibody or antigen binding fragment thereof that binds CK.

Any of the methods disclosed herein may further comprise isolating cells that are bound to the anti-EGFR antibody and/or the anti-CD105 antibody, wherein isolating the cells occurs prior to identifying cells with the first labeled antibody. The anti-EGFR antibody may be conjugated to one or more magnetic particles. The anti-CD105 antibody may be conjugated to one or more magnetic particles. The anti-EGFR antibody and the anti-CD105 antibody may be conjugated to the same magnetic particles. The anti-EGFR antibody and the anti-CD105 antibody may be conjugated to different magnetic particles. The colloidal magnetic particles may comprise ferrofluid magnetic particles. Isolating cells may comprise placing the sample in a magnetic separator. Isolating cells may comprise subjecting the sample to a magnetic field.

Any of the methods disclosed herein may comprise the use of a first and second antibody (i.e., an anti-EGFR antibody and an anti-CD105 antibody, respectively). The first antibody and the second antibody may be conjugated to one or more magnetic particles. The magnetic particles may comprise ferrofluid magnetic particles. Moreover, the first and second antibody may be conjugated to the same particle or different particles. For example, in some embodiments, magnetic enrich may comprise two populations of magnetic particles: a first population of magnetic particles decorated with anti-EGFR antibodies and a second population of magnetic particles decorated with anti-CD105 antibodies. Alternatively, in some embodiments, magnetic enrichment may employ a single population/plurality of particles that are decorated with both anti-EGFR antibodies and anti-CD105 antibodies.

Any of the methods disclosed herein may comprise isolating cells bound to a first antibody or antigen binding fragment thereof and/or a second antibody or antigen binding fragment thereof (i.e., an anti-EGFR antibody and an anti-CD105 antibody, respectively). Isolating cells may comprise subjecting the sample to a magnetic field.

The magnetic particles may be coupled to a first exogenous aggregation enhancing factor (EAEF), the first EAEF comprising one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin.

Any of the methods disclosed herein may comprise adding a second EAEF to induce aggregation of the magnetic particles, the second EAEF comprising the other member of the specific binding pair. Isolating cells bound to the first antibody or antigen binding fragment thereof and/or the second antibody or antigen binding fragment thereof (i.e., an anti-EGFR antibody and an anti-CD105 antibody, respectively) may comprise, consist of, or consist essentially of, adding to the enriched sample a member of the specific binding pair in order to reverse aggregation of the magnetic particles in the enriched sample.

Any of the methods disclosed herein may comprise adding to the sample at least one aggregation inhibiting agent selected from a group consisting of a reducing agent, an immune-complex, a chelating agent, and a diamino butane. The aggregation inhibiting agent may comprise a chelating agent. The chelating agent may comprise EDTA. The reducing agent may be mercaptoethane sulfonic acid. The aggregation inhibitor may be a bovine serum albumin (BSA).

Any of the methods disclosed herein may use a first antibody. The first antibody may be an antibody that binds to EGFR or may comprise, consist of, or consist essentially of an antigen binding fragment that binds to EGFR. Any of the methods disclosed herein may optionally use a second antibody. The second antibody may be an antibody that binds to CD105 or may comprise, consist of, or consist essentially of an antigen binding fragment that binds to CD105 protein.

Any of the methods disclosed herein may use a first labeled antibody. The first labeled antibody may comprise an antibody that binds to a CK or may comprise, consist of, or consist essentially of an antigen binding fragment that binds to a CK protein. Cytokeratins (CKs) are keratin proteins that form intermediate filaments and provide mechanical support in epithelial cells. CKs are expressed in trophoblasts at differential time points and in differential trophoblast subsets during pregnancy, with, for example, CK8, CK18, and CK19 expressed in all villous and extravillous trophoblast subsets throughout pregnancy and CK7 and CK13 expressed in syncytiotrophoblasts during the first trimester. Thus, anti-CK antibodies can be used to identify trophoblasts in a sample. Preferably, an anti-pan CK antibody that binds to more than one CK protein would be used; however, use of any anti-CK antibody for CKs that are known to be expressed by trophoblasts, such as CK7, CK8, CK13, CK18, and CK19, may be used. Anti-CK antibodies that could be used for the purposes of the present disclosure include, but are not limited to, commercially available anti-CK antibodies from Abcam (e.g., Cat. No. ab7753, Cat. No. ab86734, Cat. No. ab308262), Invitrogen (e.g., Cat. No. 53-9003-82, Cat. No. MA5-13203, Cat. No. MA5-12231, Cat. No. MA5-15507), and GeneTex (e.g., Cat. No. GTX75520, Cat. No. GTX29377, Cat. No. GTX73583). The first labeled antibody is conjugated to a label. The label may be a fluorescent label.

Any of the methods disclosed herein may use a second labeled antibody. The second labeled antibody may comprise an antibody that binds to CD45 or may comprise, consist of, or consist essentially of an antigen binding fragment that binds to a CD45 protein. CD45 is a receptor-linked protein tyrosine phosphatase that is expressed on leukocytes but are not expressed by trophoblasts. Thus, use of an anti-CD45 antibody allows for identification of cells in a sample that are not the targeted trophoblasts. Anti-CD45 antibodies that could be used for the purposes of the present disclosure include, but are not limited to, commercially available anti-CD45 antibodies from Abcam (e.g., Cat. No. ab40763, Cat. No. ab10558, Cat. No. ab281586), Invitrogen (e.g., Cat. No. 11-0459-42, Cat. No. MHCD4517, Cat. No. MA5-37809), and GeneTex (e.g., Cat. No. GTX116018, Cat. No. GTX01462-08). The second labeled antibody is conjugated to a label. The label can include a fluorescent label. The fluorescent label conjugated to the second labeled antibody may be different from the fluorescent label conjugated to the first labeled antibody.

Any of the methods disclosed herein may use a nuclear stain. The nuclear stain can be, but is not limited to, propidium iodide, DAPI, hematoxylin and cosin (H&E), and Hoechst.

The methods disclosed herein may use the first antibody (e.g., an anti-EGFR antibody) to create an enriched sample before contacting the enriched sample with the first labeled antibody and/or the second labeled antibody and/or the nuclear stain. Additionally or alternatively, the methods disclosed herein may use the first antibody (e.g., an anti-EGFR antibody) and the second antibody (e.g., an anti-CD105 antibody) to create an enriched sample before contacting the enriched sample with the first labeled antibody and/or the second labeled antibody and/or the nuclear stain.

Any of the methods disclosed herein may comprise isolating single fetal cells. This isolation process may be carried out according to any known method for isolating single cells, particularly those methods that utilize fluorescence for the purposes of sorting or isolation. Isolating single fetal cells may be based on immunofluorescent technology. Isolating single fetal cells can be carried out by fluorescence activated cell sorting (FACS). Isolating single cells can be carried out with a DEPArray. The DEPArray utilizes dielectrophoresis (DEP) to exert forces on neutral, polarizable particles, including cells. The DEPArray includes an instrument and a microfluidic cartridge, with contains a microelectronic silicon chip, microfluidic chamber and valves. The array can generate a plurality of “DEP cages” via an arrange of microelectrodes in the silicon substrate of the cartridge, where each DEP cage can capture a cell is stable levitation in the liquid to prevent contacts between the cells and surfaces of the sorting apparatus. DEP cages can also trap and move cells of different types and sizes. Once the sample containing the labeled cells are loaded in the cartridge, the cells are randomly distributed in the main chamber of the cartridge and are trapped in stable levitation in the nearest DEP cage. The trapped cells are illuminated by a stabilized LED lamp, which combines high efficiency excitation and emission filters to image the chamber in up to five fluorescent channels, allowing for detection of cells of interest. The instrument then moves the selected DEP cages with the cells of interest by changing the electric field pattern to deposit the cells in the parking chamber of the cartridge, which holds cells of interests separately from the remaining cells. The cells of interest can then be eluted from the DEPArray and collected for further analysis.

The enriched sample generated from the binding of the first antibody may be used to isolate the single fetal cells. Isolating single fetal cells can be carried out by isolating single fetal cells that are bound to the first labeled antibody and/or stained with the nuclear stain. Isolating single fetal cells can be carried out by isolating single fetal cells that are not bound to the second labeled antibody.

Any of the methods disclosed herein may comprise performing a sequencing analysis on one or more nucleic acid molecules isolated from a fetal cell. The sequencing analysis may comprise short tandem repeat (STR) analysis.

Any of the methods disclosed herein may comprise analyzing a fetal cell. Analyzing the fetal cell may comprise performing a genomic or a genetic analysis. Performing a genetic analysis may comprise detecting the presence or absence of one or more genetic abnormalities in the fetal cell. Performing a genetic analysis may comprise detecting the presence or absence of a chromosomal abnormality in the fetal cell. The chromosomal abnormality can be trisomy 21, trisomy 18, or trisomy 13.

Any of the methods disclosed herein can further comprise performing genetic testing on the fetal cell. Performing genetic testing on the fetal cell may comprise detecting the presence or absence of one or more fetal abnormalities. Performing genetic testing on the fetal cell may comprise performing a genomic analysis. Performing genetic testing on the fetal cell may comprise detecting the presence or absence of a chromosomal abnormality in the fetal cell. The chromosomal abnormality can be trisomy 21, trisomy 18, or trisomy 13.

Any of the methods disclosed herein may further comprise providing a treatment recommendation based on the results of the analysis of the fetal cell. Any of the methods disclosed may herein further comprise providing a treatment recommendation based on the results of genetic testing on the fetal cell.

Any of the methods disclosed herein may further comprise administering a therapy to the subject based on the results of the analysis of the fetal cell. Any of the methods disclosed herein may further comprise administering a therapy to the subject based on the results of genetic testing on the fetal cell. Any of the methods disclosed herein may further comprise recommending additional monitoring of the subject or fetus based on the results of the analysis of the fetal cell. Any of the methods disclosed herein may further comprise recommending additional monitoring of the subject or fetus based on the results of genetic testing on the fetal cell.

### VI. Magnetic Particles

The methods disclosed herein may comprise or use magnetic particles. For instance, any of the antibodies or antigen binding fragments disclosed herein may be conjugated to a magnetic particle. The magnetic particles may be colloidal magnetic particles. The colloidal magnetic particles may be ferrofluids.

As used herein, the term “magnetic particle” refers to a particle that can be manipulated using a magnetic field. A magnetic particle comprises a metal. Examples of metals include but are not limited to iron, nickel, cobalt, and copper.

As used herein, the term “colloidal magnetic particle” refers to a magnetic particle that is coated with a non-magnetic material. An example of a non-magnetic material is bovine serum albumin.

As used herein, the term “ferrofluid magnetic particle” refers to a colloidal magnetic particle that contains iron.

The magnetic particles can be characterized by their sub-micron particle size. The particles are generally less than about 300 nanometers (nm), 275 nm, 250 nm, 225 nm, 200 nm, 190 nm, 180 nm, 170 nm, 160 nm, 150 nm, 140 nm, 130 nm, 120 nm, 110 nm, or 100 nm in diameter. The particles are generally at least 10 nm, 20 nm, 30 nm, 40 nm, 50 nm, 60 nm, 70 nm, 80 nm, 90 nm, 100 nm, 110 nm, or 120 nm or more in diameter. The particles may be between about 40 nm to 250 nm, 40 nm to 200 nm, 50 nm to 200 nm, 50 nm to 190 nm, 50 nm to 180 nm, 50 nm to 170 nm, 60 nm to 200 nm, 70 nm to 200 nm, 80 nm to 200 nm, 90 nm to 200 nm, 90 nm to 175 nm, or 90 nm to 150 nm in diameter.

The particles may have at least 40%, 45%, 50%, 55%, 60%, 65%, 70%, 75%, 80%, 85%, 90%, 95%, or 97% or more magnetic mass. The particles may have between 40% to 95%, 45% to 95%, 50% to 90%, 55% to 90%, 60% to 90%, or 70% to 90% magnetic mass.

Particles within the range of 90-150 nm and having between 70% to 90% magnetic mass may be used.

The particles may be characterized by their resistance to gravitational separation from solution. The particles may be resistant to gravitational separation for extended periods of time. The particles may be resistant to gravitational separation for at least 1, 2, 3, 4, 5, 6, 7, 8, 9, 10, 15, 20, 25, 30, 35, 40, 45, 50, 55, 60, 75, 90, 105, or 120 or more minutes. The particles may be resistant to gravitational separation for at least 1, 2, 3, 4, 5, 6, 7, 8, 9, 10, 15, 20, 25, 30, 35, 40, 45, 50, 55, 60, 75, 90, 105, or 120 or more hours. The particles may be resistant to gravitational separation for at least 1, 2, 3, 4, 5, 6, 7, 8, 9, 10, 15, 20, 25, 30, 35, 40, 45, 50, 55, 60, 75, 90, 105, or 120 or more days.

Magnetic particles may be composed of a crystalline core of superparamagnetic material surrounded by coating molecules which are bonded, e.g., physically absorbed or covalently attached, to the magnetic core and which confer stabilizing colloidal properties. The coating material may be applied in an amount effective to prevent non-specific interactions between biological macromolecules found in the sample and the magnetic cores. Such biological macromolecules may include sialic acid residues on the surface of non-target cells, lectins, glycoproteins, and other membrane components. In addition, the coating material may contain as high a magnetic mass/nanoparticle ratio as possible. The size of the magnetic crystals comprising the core is sufficiently small that they do not contain a complete magnetic domain. The size of the nanoparticles is such that their Brownian energy exceeds their magnetic moment. Consequently, North Pole-South Pole alignment and subsequent mutual attraction/repulsion of these colloidal magnetic particles does not appear to occur even in moderately strong magnetic fields, contributing to their solution stability.

The magnetic particles may be separable in high magnetic gradient external field separators. That characteristic facilitates sample handling and provides economic advantages over the more complicated internal gradient columns loaded with ferromagnetic beads or steel wool.

Magnetic particles may be prepared by modification of base materials as described in EP0841041, which is incorporated by reference in its entirety.

Magnetic particles can be coated with antibodies or more in general any agents capable of recognizing selected proteins or antigens. Magnetic particles can be coated with any of the antibodies or agents disclosed herein. Coating of magnetic particles may be performed by any method known in the art. For instance, magnetic particles may be coated with an antibody as described in U.S. Pat. No. 6,365,362B1, which is incorporated by reference in its entirety.

An exemplary ferrofluid magnetic particle structure comprises, consists of, or consists essentially of, an iron atom surrounded by bovine serum albumin (BSA). The BSA is attached to streptavidin (SA), which is attached to biotin (BT). The BT may be attached to another BSA, which is attached to an exogenous aggregation enhancing factor (EAEF). The BT may also be attached to an antibody that binds to EGFR or CD105.

An exemplary method of magnetic particle aggregation via controlled aggregation comprises the addition to a magnetic particle coupled to a first EAEF of a second EAEF that is capable of binding to the first EAEF to promote the aggregation of the antibody-magnetic particle conjugates. The aggregation of the antibody-magnetic particle conjugates can be reversed by the addition of a third EAEF, wherein the third EAEF is capable of binding to the first EAEF or second EAEF. The third EAEF may be identical to the first EAEF. Alternatively, the third EAEF may be identical to the second EAEF. The third EAEF may be a binding partner of the first EAEF or second EAEF.

### EXAMPLES

The present technology is further illustrated by the following Examples, which should not be construed as limiting in any way.

### Example 1—Efficacy of Isolating cEVTs Using Magnetic Particles Conjugated to Anti-EGFR Antibody or Magnetic Particles Conjugated to Anti-CD105 and Anti-HER2 Antibodies

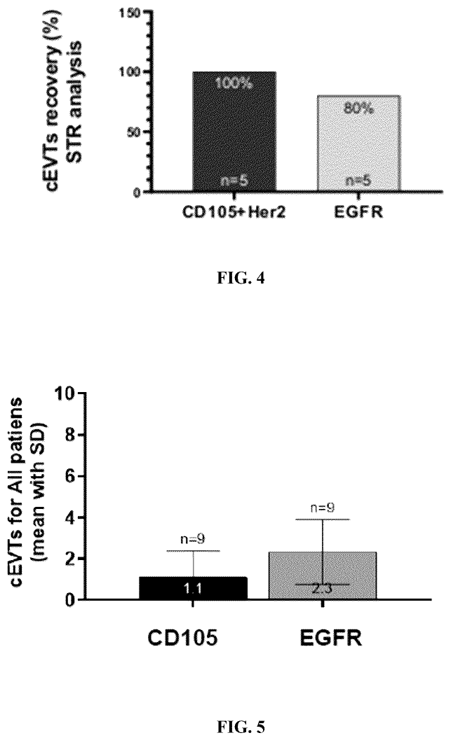

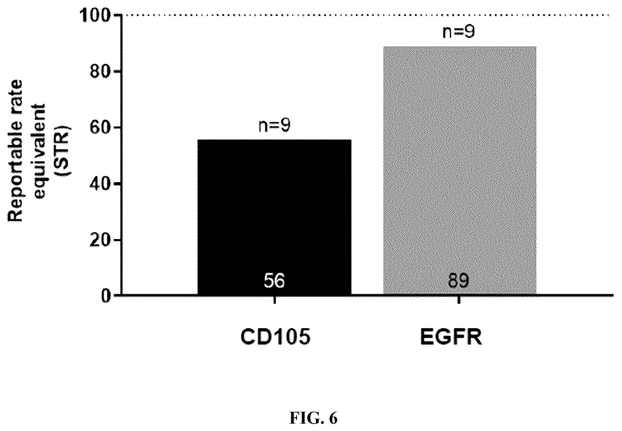

To test the efficacy of using the anti-EGFR antibody and the anti-HER2 antibody to isolate cEVTs from samples from pregnant subjects, whole blood samples were collected and subjected to either magnetic particles conjugated to HER2 and CD105 or magnetic particles conjugated to EGFR alone to generate enriched samples. The resulting enriched samples were then subjected to fluorescently labeled anti-CK antibody; and the cells bound to the anti-CK antibody was recovered using fluorescence activated cell sorting (FACS). The isolated cells were then analyzed through short tandem repeat analysis (STR) to determine if they were extravillous cytotrophoblast (cEVTs). Recovery of cEVTs was analyzed by number of cEVTs per sample for all subjects and positive subjects. The mean of cEVTs for all patients was calculated using data from both patients with no cEVTS and patients with at least 1 cEVT. The mean of cEVTs for positive patients was calculated using data from only patients with at least 1 cEVT. The magnetic particles conjugated to anti-CD105 and anti-HER2 antibodies isolated an average of 3 cEVTs in both populations (FIGS. 2 and 3). The magnetic particles conjugated to the anti-EGFR antibody isolated a mean of 2.4 cEVTs in all subjects and 3 cEVTs in positive subjects (FIGS. 2 and 3). The recovery of at least one cEVT in the analyzed patients was 100% using the magnetic particles conjugated to anti-CD105 and anti-HER2 antibodies, compared to 80% using the magnetic particles conjugated to the anti-EGFR antibody (FIG. 4). Thus, these experiments show that both the HER2-and the EGFR-based isolation techniques are capable of isolating cEVTs from samples from pregnant subjects.

Additionally, the negative fraction produced from subjecting the samples to the magnetic particles conjugated to anti-CD105 and anti-HER2 antibodies was then subjected to the magnetic particles conjugated to the anti-EGFR particle to produce a secondary enriched sample. Several cEVTs were isolated from the negative fraction using the EGFR-based isolation technique, indicating that EGFR may bind to a different subpopulation of cEVTs than HER2. This finding suggests that combinatorial use of anti-EGFR and anti-HER2 antibodies would increase the number of cEVTs recovered from a subject's sample. This may also allow for capture of differentially differentiated cEVTs that may express different markers, which would not be isolated using only one antibody or the other.

### Example 2—Efficacy of Isolating cEVTs Using Magnetic Particles Conjugated to Anti-EGFR Antibody or Magnetic Particles Conjugated to Anti-CD105 Antibody

To test the efficacy of using anti-EGFR antibody or anti-CD105 antibody to isolate cEVTs from samples derived from pregnant subjects, whole blood samples were collected and subjected to either (1) magnetic particles conjugated to anti-CD105 antibody or (2) magnetic particles conjugated to EGFR antibody to generate enriched samples. The resulting enriched samples were then subjected to fluorescently labeled anti-CK antibody; and the cells bound to the anti-CK antibody were recovered using fluorescence activated cell sorting (FACS). The isolated cells were then analyzed to determine if they were extravillous cytotrophoblast (cEVTs). Recovery of cEVTs was analyzed by number of cEVTs per sample for all subjects and positive subjects. The mean of cEVTs for all patients was calculated using data from both patients with no cEVTS and patients with at least 1 cEVT. The magnetic particles conjugated to anti-CD105 or anti-EGFR antibodies isolated an average of 1.1 or 2.3 cEVTs, respectively (FIG. 5). The recovery of at least one cEVT in the analyzed patients was 56% using the magnetic particles conjugated to anti-CD105, compared to 89% using the magnetic particles conjugated to the anti-EGFR antibody. (FIG. 6).

These results demonstrate that use of anti-EGFR antibody and anti-CD105 antibody are effective in isolating cEVTs from maternal blood samples. The findings further suggest that the use of anti-EGFR antibody would increase the number of cEVTs recovered from a patient sample relative to the use of anti-CD105 antibody.

### Example 3—Efficacy of Isolating cEVTs Using a Mixture of Magnetic Particles Conjugated to Anti-CD105 Antibody and Magnetic Particles Conjugated to Anti-EGFR Antibody or Magnetic Particles Conjugated to Anti-CD105 Antibody Alone

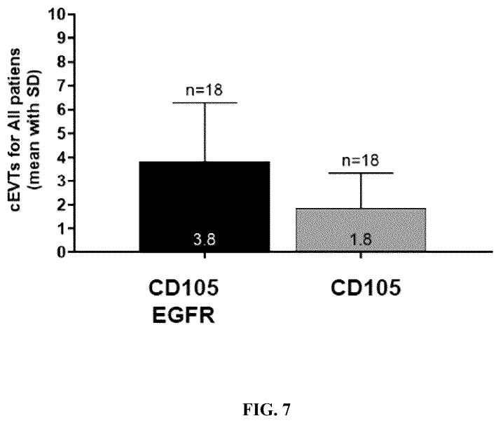

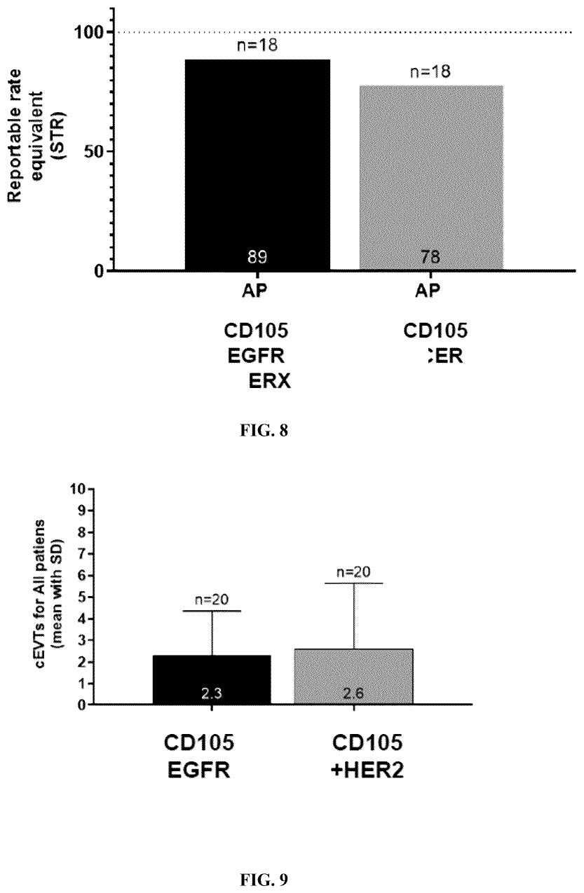

To test the efficacy of anti-EGFR antibody and anti-CD105 antibody to isolate cEVTs from samples derived from pregnant subjects, whole blood samples were collected and subjected to either (1) a mixture of magnetic particles conjugated to anti-CD105 antibody and magnetic particles conjugated to anti-EGFR antibody, or (2) magnetic particles conjugated to anti-CD105 antibodies alone to generate enriched samples. The resulting enriched samples were then subjected to fluorescently labeled anti-CK antibody; and the cells bound to the anti-CK antibody were recovered using fluorescence activated cell sorting (FACS). The isolated cells were then analyzed to determine if they were extravillous cytotrophoblast (cEVTs). Recovery of cEVTs was analyzed by number of cEVTs per sample for all subjects and positive subjects. The mean of cEVTs for all patients was calculated using data from both patients with no cEVTS and patients with at least 1 cEVT. The mean of cEVTs for positive patients was calculated using data from only patients with at least 1 cEVT. The mixture of magnetic particles conjugated to anti-CD105 antibody and magnetic particles conjugated to anti-EGFR antibody isolated an average of 3.8 cEVTs, while magnetic particles conjugated to the anti-CD105 antibody alone isolated an average of 1.8 cEVTs (FIG. 7). The recovery of at least one cEVT in the analyzed patients was 89% using the magnetic particles conjugated to anti-CD105 and anti-EGFR antibodies, compared to 78% using the magnetic particles conjugated to the anti-CD105 antibody. (FIG. 8).

These findings demonstrated that the use of a mixture of magnetic particles conjugated to anti-EGFR antibody or anti-CD105 increases the number of cEVTs recovered from a patient sample relative to the use of anti-CD105 antibody alone.

### Example 4—Efficacy of Isolating cEVTs Using a Mixture of Magnetic Particles Conjugated to Anti-CD105 Antibody and Magnetic Particles Conjugated to Anti-EGFR Antibody or a Mixture of Magnetic Particles Conjugated to Anti-CD105 Antibody and Magnetic Particles Conjugated to Anti-HER2 Antibody

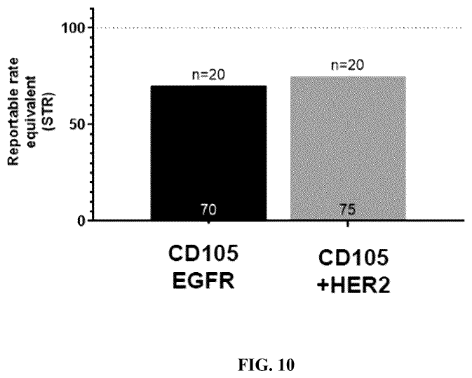

To assess the efficacy of anti-CD105 antibody in combination with anti-EGFR antibody or anti-HER2 antibody to isolate cEVTs from samples derived from pregnant patients, whole blood samples were collected and subjected to either (1) a mixture of magnetic particles conjugated to anti-CD105 antibody and magnetic particles conjugated to anti-EGFR antibody or (2) a mixture of magnetic particles conjugated to anti-CD105 antibody or anti-HER2 antibody to generate enriched samples. The resulting enriched samples were then subjected to fluorescently labeled anti-CK antibody; and the cells bound to the anti-CK antibody were recovered using fluorescence activated cell sorting (FACS). The isolated cells were then analyzed to determine if they were extravillous cytotrophoblast (cEVTs). Recovery of cEVTs was analyzed by number of cEVTs per sample for all patients. The mean of cEVTs for all patients was calculated using data from both patients with no cEVTS and patients with at least 1 cEVT. The mixture of magnetic particles conjugated to anti-CD105 antibody and magnetic particles conjugated to anti-EGFR antibody isolated an average of 2.3 cEVTs, while mixture of magnetic particles conjugated to anti-CD105 antibody or anti-HER2 antibody isolated an average of 2.6 cEVTs (FIG. 9). The recovery of at least one cEVT in the analyzed patients was 70% using the magnetic particles conjugated to anti-CD105 and anti-EGFR antibodies, compared to 75% using the magnetic particles conjugated to the anti-CD105 antibody or anti-HER2 antibody. (FIG. 10). These findings demonstrated that the use of anti-CD105 and anti-EGFR antibodies conjugated to separate magnetic particles increases the number of cEVTs recovered from a patient sample similar to the mixture of anti-CD105 and anti-HER2 antibodies conjugated to separate magnetic particles.

### Example 5—Efficacy of Isolating cEVTs Using Magnetic Particles Conjugated to Anti-CD105 Antibody and Anti-EGFR Antibody or a Mixture of Magnetic Particles Conjugated to Anti-CD105 Antibody and Magnetic Particles Conjugated to Anti-EGFR Antibody

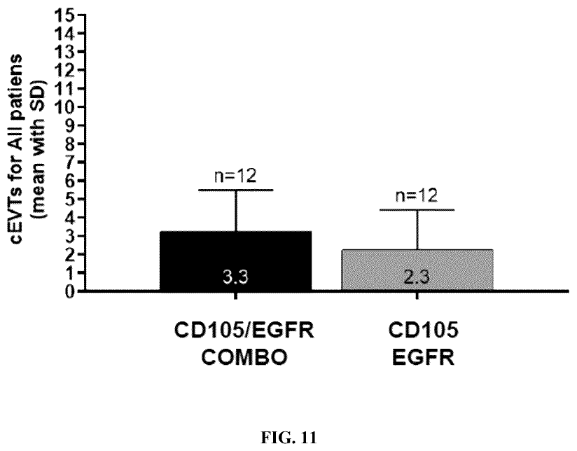

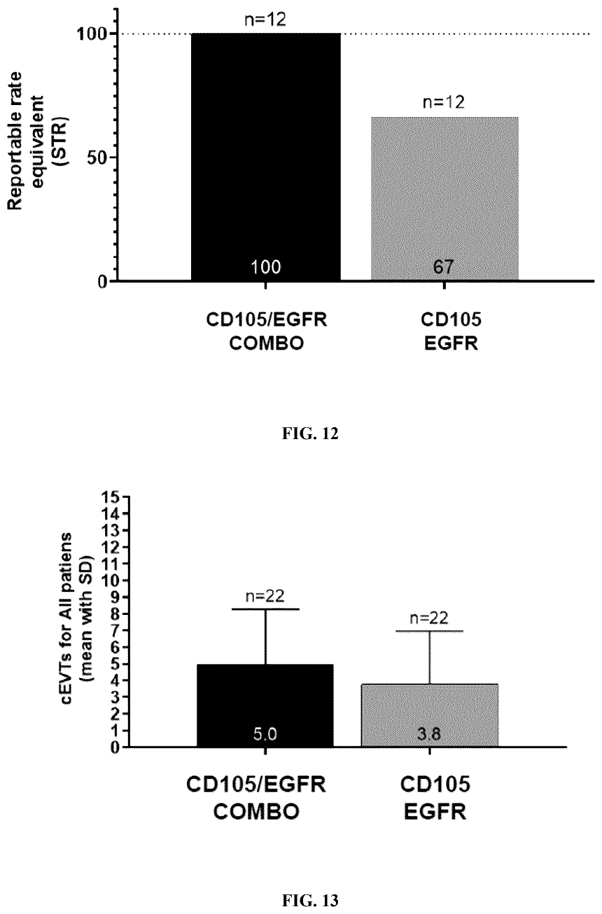

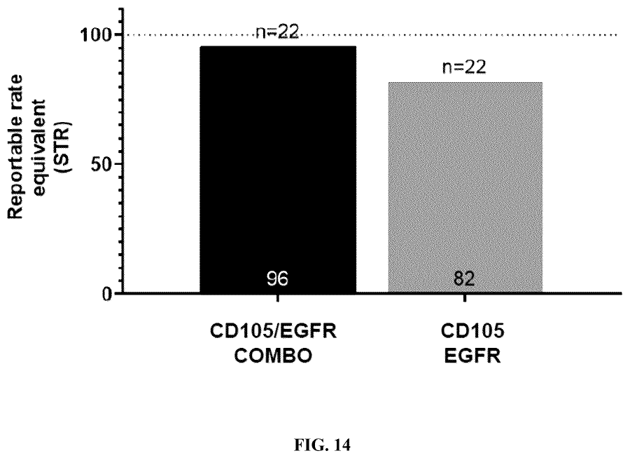

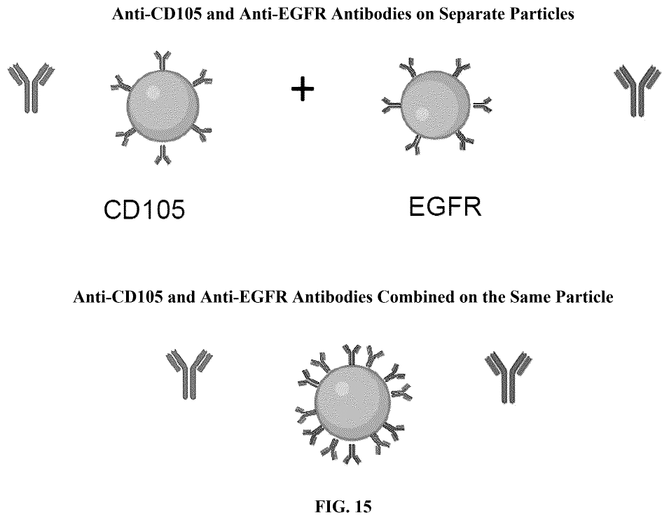

To assess the efficacy of the combinatorial use of anti-CD105 and anti-EGFR antibodies to isolate cEVTs from samples derived from pregnant patients, whole blood samples were collected and subjected to either (1) magnetic particles conjugated to anti-CD105 antibody and anti-EGFR antibody or (2) a mixture of magnetic particles conjugated to anti-CD105 antibody and magnetic particles conjugated to anti-EGFR antibody to generate enriched samples. Whole blood samples consisting of 20 or 30 mL of maternal blood were used for the cEVT isolation studies. The resulting enriched samples were then subjected to fluorescently labeled anti-CK antibody; and the cells bound to the anti-CK antibody were recovered using fluorescence activated cell sorting (FACS). The isolated cells were then analyzed to determine if they were extravillous cytotrophoblast (cEVTs). Recovery of cEVTs was analyzed by number of cEVTs per sample for all patients. The mean of cEVTs for all patients was calculated using data from both patients with no cEVTS and patients with at least 1 cEVT. Magnetic particles conjugated to anti-CD105 and anti-EGFR antibodies isolated an average of 3.3 cEVTs from 20 mL of maternal blood, while a mixture of magnetic particles conjugated to anti-CD105 antibody and magnetic particles conjugated to anti-EGFR antibody isolated an average of 2.3 cEVTs (FIG. 11). Magnetic particles conjugated to anti-CD105 and anti-EGFR antibodies isolated an average of 5.0 cEVTs from 30 mL of maternal blood, while a mixture comprising magnetic particles conjugated to anti-CD105 antibody and magnetic particles conjugated to anti-EGFR antibody isolated an average of 3.8 cEVTs (FIG. 13). Using 20 mL maternal blood, the recovery of at least one cEVT in the analyzed patients was 100% using the magnetic particles conjugated to both anti-CD105 and anti-EGFR antibodies, compared to 67% using the mixture comprising magnetic particles conjugated to anti-CD105 antibody and magnetic particles conjugated to anti-EGFR antibody (FIG. 12). With 30 mL of maternal blood, the recovery of at least one cEVT in the analyzed patients was 96% using the magnetic particles conjugated to both anti-CD105 and anti-EGFR antibodies, compared to 82% using the mixture comprising magnetic particles conjugated to anti-CD105 antibody and magnetic particles conjugated to anti-EGFR antibody (FIG. 14). These findings demonstrated that the combinatorial use of anti-CD105 and anti-EGFR antibodies conjugated to magnetic particles increases the number of cEVTs recovered from a patient sample relative to a mixture of magnetic particles conjugated to anti-CD105 antibody or anti-EGFR antibody.

Thus, these experiments show that both the CD105 and the EGFR mixture and combinatorial based isolation techniques are capable of isolating cEVTs in samples derived from pregnant subjects.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds, compositions or biological systems, which can, of course, vary. It is also to be understood that the terminology used herein is for the purpose of describing particular embodiments only and is not intended to be limiting.

All patents, patent applications, provisional applications, and publications referred to or cited herein are incorporated by reference in their entirety, including all figures and tables, to the extent that are not inconsistent with the explicit teachings of this specification.

1. A method for isolating fetal cells from a sample from a pregnant subject, comprising:
(a) contacting the sample comprising a plurality of cells with a plurality of magnetic particles, the magnetic particles conjugated to an anti-epidermal growth factor receptor (EGFR) antibody;
(b) isolating cells bound to the anti-EGFR antibody by subjecting the sample to a magnetic field, thereby producing an enriched sample;
(c) contacting the enriched sample with a first labeled antibody that binds to a cytokeratin (CK); and
(d) isolating a fetal cell that is bound to the first labeled antibody;
wherein the fetal cell is a fetal nucleated red blood cell (fnRBC) or a trophoblast.

2-3. (canceled)

4. The method of claim 1, wherein the trophoblast is an extravillous cytotrophoblast (cEVT).

5. The method of claim 1, wherein the magnetic particles are colloidal magnetic particles.

6. (canceled)

7. The method of claim 1, wherein the magnetic particles are coupled to a first exogenous aggregation enhancing factor (EAEF), the first EAEF comprising one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin;
wherein (a) comprises adding a second EAEF to induce aggregation of the magnetic particles, and wherein the second EAEF comprises the other member of the specific binding pair; and
wherein (b) comprises adding to the enriched sample a member of the specific binding pair in order to reverse aggregation of the magnetic particles in the enriched sample.

8-9. (canceled)

10. The method of claim 1, wherein (c) further comprises contacting the enriched sample with a second labeled antibody that binds to CD45, wherein the second labeled antibody comprises a fluorescent label

11-15. (canceled)

16. A method for isolating fetal cells from a sample from a pregnant subject, comprising:
(a) contacting the sample comprising a plurality of cells with a plurality of magnetic particles, wherein each magnetic particle in the plurality is conjugated to an anti-EGFR antibody;
(b) subjecting the sample to a magnetic field;
(c) isolating cells bound to the magnetic particles to produce an enriched sample;
(d) contacting the enriched sample with:
(i) an antibody that binds to a cytokeratin (CK) and is conjugated to a fluorescent label,
(ii) an antibody that binds to CD45 and is conjugated to a fluorescent label, and
(iii) a nuclear stain; and
(d) isolating a fetal cell using fluorescence activated cell sorting (FACS) or DEPArray, wherein the fetal cell is positive for the CK and nuclear stain and negative for CD45;
wherein the fetal cell is a fetal nucleated red blood cell (fnRBC) or a trophoblast.

17-18. (canceled)

19. The method of claim 16, wherein the trophoblast is an extravillous cytotrophoblast (cEVT).

20. The method of claim 16, wherein the magnetic particles are colloidal magnetic particles.

21. (canceled)

22. The method of claim 16, further comprising genetic sequencing of the fetal cell or performing a genomic or genetic analysis of the fetal cell.

23-33. (canceled)

34. A method for isolating fetal cells from a sample from a pregnant subject, comprising:
(a) contacting the sample comprising a plurality of cells with a plurality of magnetic particles, wherein the plurality of magnetic particles comprises:
(i) magnetic particles that are conjugated to an anti-epidermal growth factor receptor (EGFR) antibody and an anti-CD105 antibody, or
(ii) a combination of magnetic particles that are conjugated to an anti-EGFR antibody and magnetic particles that are conjugated to an anti-CD105 antibody;
(b) isolating cells bound to the first antibody or the second antibody by subjecting the sample to a magnetic field, thereby producing an enriched sample;
(c) contacting the enriched sample with a first labeled antibody that binds to a cytokeratin (CK); and
(d) isolating a fetal cell that is bound to the first labeled antibody;
wherein the fetal cell is a fetal nucleated red blood cell (fnRBC) or a trophoblast.

35-36. (canceled)

37. The method of claim 34, wherein the trophoblast is an extravillous cytotrophoblast (cEVT).

38. The method of claim 34, wherein the magnetic particles are colloidal magnetic particles.

39. (canceled)

40. The method of claim 34, wherein the magnetic particles are coupled to a first exogenous aggregation enhancing factor (EAEF), the first EAEF comprising one member of a specific binding pair selected from the group comprising biotin-streptavidin, antigen-antibody, receptor-hormone, receptor-ligand, agonist-antagonist, lectin-carbohydrate, Protein A-antibody Fc, and avidin-biotin, biotin analog-avidin, desthiobiotin-streptavidin, desthiobiotin-avidin, iminobiotin-streptavidin, and iminobiotin-avidin;
wherein (a) comprises adding a second EAEF to induce aggregation of the magnetic particles. and wherein the second EAEF comprises the other member of the specific binding pair; and
wherein (b) comprises adding to the enriched sample a member of the specific binding pair in order to reverse aggregation of the magnetic particles in the enriched sample.

41-42. (canceled)

43. The method of claim 34, wherein (c) further comprises contacting the enriched sample with a second labeled antibody that binds to CD45, wherein the second labeled antibody comprises a fluorescent label.

44-45. (canceled)

46. The method of claim 34, wherein isolating the fetal cell comprises using immunofluorescent technology, fluorescence activated cell sorting (FACS), or DEP Array.

47. The method of claim 34, further comprising gene sequencing of the fetal cell or performing a genomic or genetic analysis of the fetal cell.

48. (canceled)

49. A method for isolating fetal cells from a sample from a pregnant subject, comprising:
(a) contacting the sample comprising a plurality of cells with a plurality of magnetic particles, wherein the plurality of magnetic particles comprises:
(i) magnetic particles that are conjugated to an anti-epidermal growth factor receptor (EGFR) antibody and an anti-CD105 antibody, or
(ii) a combination of magnetic particles that are conjugated to an anti-EGFR antibody and magnetic particles that are conjugated to an anti-CD105 antibody;
(b) subjecting the sample to a magnetic field;
(c) isolating cells bound to the magnetic particles to produce an enriched sample;
(d) contacting the enriched sample with:
(i) an antibody that binds to a cytokeratin (CK) and is conjugated to a fluorescent label,
(ii) an antibody that binds to CD45 and is conjugated to a fluorescent label, and
(iii) a nuclear stain; and
(d) isolating a fetal cell using fluorescence activated cell sorting (FACS) or DEPArray, wherein the fetal cell is positive for the CK and nuclear stain and negative for CD45;
wherein the fetal cell is a fetal nucleated red blood cell (fnRBC) or a trophoblast.

50-51. (canceled)

52. The method of claim 49, wherein the trophoblast is an extravillous cytotrophoblast (cEVT).

53. The method of claim 49, wherein the magnetic particles are colloidal magnetic particles.

54. (canceled)

55. The method of claim 49, further comprising genetic sequencing of the fetal cell or performing a genomic or genetic analysis of the fetal cell.

56. (canceled)

57. A method for isolating fetal cells from a sample from a pregnant subject, comprising:
(a) contacting the sample comprising an anti-epidermal growth factor receptor (EGFR) antibody and an anti-CD105 antibody;
(b) isolating cells bound to the anti-EGFR antibody and/or anti-CD105 antibody, thereby producing an enriched sample;
(c) contacting the enriched sample with a first labeled antibody that binds to a fetal cell marker; and
(d) isolating a fetal cell that is bound to the first labeled antibody;
wherein the fetal cell is a fetal nucleated red blood cell (fnRBC) or a trophoblast.

58. The method of claim 57, wherein the fetal cell marker is a cytokeratin (CK).

59. (canceled)

60. The method of claim 57, wherein the anti-EGFR antibody and the anti-CD105 antibody are each independently conjugated to a colloidal magnetic particle.

61-62. (canceled)

63. The method of claim 57, wherein (c) further comprises contacting the enriched sample with a second labeled antibody that binds to CD45.

64. The method of claim 57, wherein isolating the fetal cell comprises using immunofluorescent technology, fluorescence activated cell sorting (FACS), or DEPArray.

65. The method of claim 57, further comprising gene sequencing of the fetal cell or performing a genomic or genetic analysis of the fetal cell.

66. (canceled)
